SEQUENCE LISTING (4 sequences)

SEQ ID NO: 1 - protein, 30 aa, Artificial Sequence
  peptide 1
GDGPVQGIINFEQKESNGPVKVWGSIKGLT

SEQ ID NO: 2 - protein, 39 aa, Artificial Sequence
  peptide 2
ATKAVCVLKGDGPVQGIINFEQKESNGPVKVWGSIKGLT

SEQ ID NO: 3 - protein, 90 aa, Artificial Sequence
  peptide 3
ATKAVCVLKGDGPVQGIINFEQKESNGPVKVWGSIKGLTEGLHGFHVHEFGDNTAGCTSAGPHFNPLSRKHGGPKDEERHVGDLGNVTAD

SEQ ID NO: 4 - protein, 90 aa, Artificial Sequence
  peptide 4
ATKAVCVLKGDGPVQGIINFEQKESNGPVKVWGSIKGLTEGLHGFHVHEFGDNTAGCTSAGPHFNPLSRKHGGPKDEERHVGDLRNVTAD

(19) United States
(12) Patent Application Publication
(10) Pub. No.: US 2023/0183661 A1
(43) Pub. Date: Jun. 15, 2023

(54) BICOMPATIBLE PEPTIDEBIOCOMPATIBLE PEPTIDES FOR INHIBITION OF AGGREGATION OF B-AMYLOID PROTEIN

(71) Applicant: Catholic University Industry Academic Cooperation Foundation, Seoul (KR)

(72) Inventors: Hyang Shuk RHIM, Seoul (KR); Seong Man KANG, Seoul (KR); Ja Young JANG, Seoul (KR)

(21) Appl. No.: 16/071,340

(22) PCT Filed: Mar. 29, 2018
(86) PCT No.: PCT/KR2018/003709

§ 371 Date: Jul. 19, 2018

(30) Foreign Application Priority Data
May 8, 2017 (KR) 1020170057419

(51) Int. Cl. C12N 9/02; A61P 25/28; C12N 15/52; A61K 38/44

(52) U.S. Cl. CPC C12N 9/0089; C12Y 115/01001; A61P 25/28; C12N 15/52; A61K 38/446

(57) ABSTRACT

The present invention relates to a biocompatible peptide inhibiting the aggregation of a β-amyloid protein, and more particularly, to a biocompatible peptide derived from superoxide dismutase 1 (SOD1) and specifically binding to β-amyloid, a β-amyloid aggregation inhibitor including the peptide, and a pharmaceutical composition for treating a β-amyloid aggregation-associated disease. The peptide of the present invention has a strong binding strength to β-amyloid, inhibits the formation of a β-amyloid protein aggregate, and prevents neural cell death, thereby treating a neurodegenerative disease such as Alzheimer's disease. In addition, since the peptide of the present invention does not have cytotoxicity, it has no side effect of disturbing an immune system.

### SEQUENCE LISTINGS

The sequence listings contained in the electronic file titled “SOP 114163US_sequence_listing.txt,” with a creation date of 9 Jan. 2021, comprising 5 kB, hereby incorporated herein, are identical to the sequence listings disclosed and described herein.

### TECHNICAL FIELD

The present invention relates to a peptide that inhibits the aggregation of a β-amyloid protein, and more particularly, to a biocompatible peptide derived from superoxide dismutase 1 (SOD1) and specifically binding to β-amyloid, a β-amyloid aggregation inhibitor including the peptide, and a pharmaceutical composition for treating a β-amyloid aggregation-associated disease.

### BACKGROUND ART

Alzheimer's disease (AD) is the most common neurodegenerative disease and is fatal. Although having been intensively studied for over 100 years, there is still no treatment method, and AD is a fatal disease leading to death within 10 years after the onset of the disease.

Amyloid plaques are extracellular deposits of β-amyloid (Aβ) and are the cause of a neurodegenerative disease.

Generally, an extracellular aggregate of an Aβ protein has been known to have a critical function for the pathological phenomenon of AD. However, according to a recent study, it has been revealed that when Aβ is deposited in nerve cells, the intraneuronal Aβ causes the formation of an extracellular aggregate of an Aβ protein. Through the result of such a study, it can be seen that extraneuronal Aβ and an intraneuronal aggregate of an Aβ protein have to be removed from the brain of an Alzheimer patient to treat AD.

Today, to treat AD, there are i) a method for reducing Aβ production, ii) a method for inhibiting an Aβ protein aggregate, iii) a method for regulating the enzyme activity of a secretase involved in Aβ formation and iv) a method for removing an Aβ plaque using Aβ antibodies.

Such therapeutic methods have a problem that often causes side effects such as deterioration of cognitive ability and a change in the immune system. In addition, the regulation of such enzyme activity induces the deposition of a different fragment protein (Aβ peptide) and interferes with cell homeostasis including neuronal activity.

To solve the above problems, it is necessary to identify a specific cellular Aβ-binding protein and comprehend a molecular biological mechanism that inhibits the formation of an Aβ protein aggregate, and side effects resulting from a decrease in Aβ protein need to be prevented. In this aspect, an immunotherapy based on a specific Aβ protein has been widely studied in clinical trials, and is evaluated as a promising therapeutic method for AD.

### DISCLOSURE

### Technical Problem

The present invention is directed to providing a biocompatible peptide that is derived from SOD1 and specifically binds to Aβ.

The present invention is also directed to providing an Aβ aggregation inhibitor including the peptide.

The present invention is also directed to providing an expression vector including the peptide and host cells transformed with the expression vector.

The present invention is also directed to providing a method for producing a protein including the peptide by culturing the transformed host cells.

### Technical Solution

The present invention has been conceived to solve the above-mentioned problems, and the present invention provides a biocompatible peptide that is derived from SOD1 and specifically binds to Aβ.

According to an exemplary embodiment of the present invention, the peptide may include the β2-to-β3 amino acid sequence of SOD1.

According to another exemplary embodiment of the present invention, the peptide may include the β1-to-β3 amino acid sequence of SOD1.

According to still another exemplary embodiment of the present invention, the peptide may include the β1-to-β5 amino acid sequence of SOD1.

According to yet another exemplary embodiment of the present invention, the peptide may consist of one or more amino acids selected from the group of amino acid sequences of SEQ ID NOs: 1 to 4.

According to yet another exemplary embodiment of the present invention, the peptide may not form a self-aggregate.

According to yet another exemplary embodiment of the present invention, the peptide may not have cytotoxicity.

According to yet another exemplary embodiment of the present invention, the peptide may interrupt β-sheet interactions between Aβs.

According to yet another exemplary embodiment of the present invention, the peptide may inhibit Aβ aggregation.

In addition, the present invention provides an Aβ aggregation inhibitor including a peptide that is derived from SOD1 and specifically binds to Aβ.

In addition, the present invention provides a pharmaceutical composition for treating an Aβ aggregation-associated disease, which includes a peptide that is derived from SOD1 and specifically binds to Aβ.

In addition, the present invention provides a pharmaceutical composition for treating a neurodegenerative disease, which includes a peptide that is derived from SOD1 and specifically binds to Aβ.

According to an exemplary embodiment of the present invention, the neurodegenerative disease may be any one or more selected from the group consisting of amyotrophic lateral sclerosis (Lou Gehrig's disease), Parkinson's disease, AD (type III diabetes), Huntington's disease, spinocerebellar degeneration and type II diabetes.

In addition, the present invention provides an expression vector including a polynucleotide encoding a peptide that is derived from SOD1 and specifically binds to Aβ.

According to an exemplary embodiment of the present invention, the peptide may include the FLAG-tag nucleic acid sequence bound to the N-terminus and the myc-tag sequence bound to the C-terminus.

According to another exemplary embodiment of the present invention, the peptide may include the EGFP tag sequence bound to the N-terminus.

According to still another exemplary embodiment of the present invention, the peptide may include a glutathione S-transferase (GST)-tag sequence bound to the N-terminus.

In addition, the present invention provides host cells that are transformed with any one of the expression vectors.

According to an exemplary embodiment of the present invention, the host cells may be any one selected from the group consisting of human embryonic kidney cells (HEK293T), mouse neuroblastoma cells (Neuro 2A, N2a) and glioma cells (neuroglioma H4).

In addition, the present invention provides a method for producing a peptide specifically binding to Aβ. The method for producing a peptide may include:

(A) preparing an expression vector which includes a polynucleotide encoding a peptide specifically binding to Aβ;

(B) preparing a transformant by introducing the expression vector into host cells; and

(C) inducing expression of the peptide by culturing the transformant and obtaining the peptide.

According to an exemplary embodiment of the present invention, the peptide may be derived from SOD1.

According to another exemplary embodiment of the present invention, the peptide may include the β2-to-β3 amino acid sequence of SOD1.

According to still another exemplary embodiment of the present invention, the peptide may include the β1-to-β3 amino acid sequence of SOD1.

According to yet another exemplary embodiment of the present invention, the peptide may include the β1-to-β5 amino acid sequence of SOD1.

According to yet another exemplary embodiment of the present invention, the peptide may consist of one or more amino acids selected form the group of amino acid sequences of SEQ ID NOs: 1 to 4.

### Advantageous Effects

A peptide of the present invention strongly binds to Aβ, inhibits the formation of an aggregate of an Aβ protein and prevents neural cell death, and thus a neurodegenerative disease such as AD can be treated. In addition, the peptide of the present invention has no cytotoxicity and has no side effect of disturbing an immune system.

### DESCRIPTION OF DRAWINGS

FIG. 1 is a schematic diagram showing the mechanism by which a peptide (natural Aβ Binder and Aβ aggregation inhibitor; NABi) of the present invention inhibits Aβ aggregation, resulting in inhibition of neural cell death.

FIG. 2 is a schematic diagram showing the structure of SOD1 from which a peptide of the present invention is derived and the β1-to-β8 structure thereof.

FIG. 3 is a result of identifying a determinant of a peptide (NABi) of the present invention, which binds to Aβ.

FIG. 4 is a result of an experiment showing that a peptide (NABi) of the present invention does not form a self-aggregate.

FIG. 5 is a result of an immunofluorescence assay (IFA) experiment confirming that a peptide (NABi) of the present invention has no cytotoxicity.

FIG. 6 is a result of an immunoblotting (IB) experiment confirming that a peptide (NABi) of the present invention has no cytotoxicity.

FIG. 7 is a result of an IFA experiment confirming that a peptide (NABi) of the present invention inhibits Aβ aggregation.

FIG. 8 is a result of an IB experiment confirming that a peptide (NABi) of the present invention inhibits Aβ aggregation.

FIG. 9 is a result of a fluorescence recovery after photobleaching (FRAP) assay experiment verifying mobility of a peptide (NABi) of the present invention while binding to Aβ.

FIG. 10 is an experimental result confirming that a peptide (NABi) of the present invention inhibits neural cell death through PI staining.

FIG. 11 is an experimental result confirming that a peptide (NABi) of the present invention inhibits neural cell death through nuclear morphology.

FIG. 12 is an experimental result confirming that neural cell death is inhibited according to the concentration of a peptide (NABi) of the present invention through AnnexinV staining.

FIG. 13 is a result of evaluating that a peptide (NABi) of the present invention inhibits neural cell death at a molecular level.

### MODES OF THE INVENTION

Hereinafter, the present invention will be described in further detail.

### Definition of Terms

The term “NABi” used herein is an abbreviation of Natural Amyloid β Binder and Amyloid β aggregation inhibitor, and refers to a protein consisting of the β1-to-β5 strand sequence of a SOD1 protein. The β1-to-β5 strand sequence may consist of an amino acid sequence of SEQ ID NO: 1 or 2, or an amino acid sequence homologous with SEQ ID NO: 1 or 2.

The term “neurodegenerative disease” used herein includes all conditions which degenerate nerve cells, but generally does not include conditions that definitely result from factors other than nerve cells, such as cerebrovascular disorders, trauma, metabolic disorders, etc., and is an umbrella term for diseases causing neurodegeneration, for example, AD, Parkinson's disease, amyotrophic lateral sclerosis (Lou Gehrig's disease), a polyglutamine disease (Huntington's disease), and spinocerebellar degeneration.

The term “β-amyloid” used herein refers to beta amyloid, amyloid beta or Aβ, and generally a peptide consisting of 36 to 43 amino acids.

The term “expression vector” used herein is a linear or circular DNA molecule that consists of a fragment encoding a polypeptide of interest and is operably linked to additional fragments provided for transcription of the expression vector. Such additional fragments include a promoter and a termination coding sequence. An expression vector also includes one or more replication origins, one or more selection markers, and a polyadenylation signal. The expression vector is generally derived from plasmid or viral DNA, or contains both factors.

In the expression vector according to the present invention, the expression vector may be a plasmid, a virus vector, a phage particle, or a genome insert. The expression vector may be transformed into host cells, and then replicated regardless of the genome of the host cells or integrated into the genome of the host cells.

The term “transformation” or “transfection” used herein refers to introduction of DNA into a host to become replicated as an extrachromosomal factor or by chromosome integration. A method for transforming an expression vector according to the present invention may be electroporation, a calcium phosphate (CaPO₄) method, a calcium chloride (CaCl₂) method, microinjection, a polyethylene glycol (PEG) method, a DEAE-dextran method, a cationic liposome method or a lithium acetate-DMSO method, but the present invention is not limited thereto.

In host cells according to the present invention, the host cells are preferably host cells with high DNA introduction efficiency and high expression efficiency of the introduced DNA, and all microorganisms including prokaryotes and eukaryotes may be used.

Amino Acid Sequence of Peptide of the Present Invention

A peptide of the present invention includes the β2-to-β3, β1-to-β3 or β1-to-β5 amino acid sequence of SOD1, and the peptide may include fragments or variants of amino acids having different sequences by deletion, insertion, substitution of an amino acid residue or a combination thereof without affecting the function of a protein.

Amino acid exchange, at protein and peptide levels, which does entirely change the activity of the peptide is known in the art. In this case, the modification of an amino acid may be accomplished by phosphorylation, sulfation, acrylation, glycosylation, methylation, or farnesylation.

Therefore, the peptide of the present invention includes a peptide including the β2-to-β3, β1-to-β3 or β1-to-β5 amino acid sequence of SOD1, a peptide having the substantially the same amino acid sequence, and a variant or active fragment thereof. The substantially the same peptide refers to a peptide having at least 80%, preferably at least 90%, and most preferably at least 95% homology with the above-mentioned amino acid sequence, but the present invention is not limited thereto, and if a peptide has at least 80% homology with the amino acid sequence and exhibits the same activity, it is included in the scope of the present invention.

The β2-to-β3 amino acid sequence of SOD1 may be SEQ ID NO: 1, the β1-to-β3 amino acid sequence of SOD1 may be SEQ ID NO: 2, and the β1-to-β5 amino acid sequence of SOD1 may be SEQ ID NO: 3 or 4. The amino acid sequence of SEQ ID NO: 1 to 4 is shown in Table 1 below. SEQ ID NO: 3 is a wild type, and SEQ ID NO: 4 is a mutant type (G85R).

TABLE 1
Amino acid sequences of SEQ ID NOs: 1 to 4

| | |
|---|---|
| SEQ ID NO: 1 | gdgpvqgiin feqkesngpv kvwgsikglt |
| SEQ ID NO: 2 | atkavcvlk gdgpvqgiin feqkesngpv |
| | kvwgsikglt |
| SEQ ID NO: 3 | atkavcvlkg dgpvqgiinf eqkesngpvk |
| | vwgsikglte glhgfhvhef gdntagctsa |
| | gphfnplsrk hggpkdeerh vgdlgnvtad |
| SEQ ID NO: 4 | atkavcvlkg dgpvqgiinf eqkesngpvk |
| | vwgsikglte glhgfhvhef gdntagctsa |
| | gphfnplsrk hggpkdeerh vgdl r nvtad |

The peptide (NABi) of the present invention is based on SOD1 and the structural characteristic of a β-sheet of Aβs, and the β1 to β5 of SOD1 was identified based on a determinant of SOD1 binding to Aβ. It was named NABi.

It was confirmed that the peptide (NABi) of the present invention effectively inhibits an Aβ protein aggregate by blocking intermolecular β-sheet interactions between Aβ proteins. It was confirmed that Aβ-associated neural cell death is inhibited by inhibiting the formation of an Aβ protein aggregate.

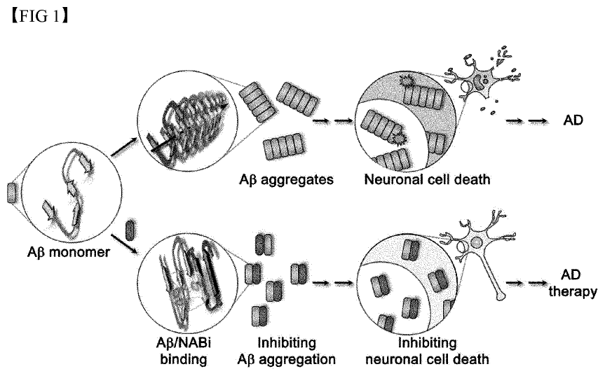

FIG. 1 schematically illustrates a mechanism by which the peptide (NABi) of the present invention inhibits Aβ protein aggregate formation to inhibit Aβ-associated neural cell death, thereby treating AD.

The peptide (NABi) of the present invention is derived from a protein in a living body, and does not form a self-aggregate as an intermolecular β-sheet breaker and has no cytotoxicity. Therefore, the peptide can be developed as a safe and new therapeutic agent for a neurodegenerative disease.

In addition, the peptide (NABi) of the present invention can be used as a therapeutic agent for AD, which is known as type III diabetes (brain diabetes). Moreover, the peptide can also be used as a therapeutic agent for other neurodegenerative diseases that occur due to the formation of an Aβ protein aggregate, for example, Lou Gehrig's disease (ALS), Parkinson's disease and Huntington's disease.

A therapeutically effective amount of the composition of the present invention may vary according to various factors, for example, an administration method, a target site, a patient's condition, etc. Therefore, when the composition is used in a human body, a dosage has to be determined as a suitable amount in consideration of safety and efficiency. It is possible to estimate an amount used in humans from the effective amount determined by an animal test. Such things that should be considered to determine an effective amount are described, for example, in Hardman and Limbird, eds. Goodman and Gilman's The Pharmacological Basis of Therapeutics, 10th ed. (2001), Pergamon Press; and E. W. Martin ed., Remington's Pharmaceutical Sciences, 18th ed. (1990), Mack Publishing Co.

The composition of the present invention may also include a carrier, a diluent, an excipient, which are conventionally used in a biological preparation, or a combination thereof. A pharmaceutically acceptable carrier may be any one that is suitable for delivering the composition into a living body without particular limitation, and the pharmaceutically acceptable carrier may be, for example, a compound described in the Merck Index, 13th ed. Merck & Co. Inc., saline, distilled water, Ringer's solution, buffered saline, a dextrose solution, a maltodextrin solution, glycerol, ethanol, or a mixture of one or more thereof. Another conventional additive such as an antioxidant, a buffer, or a bacteriostat may be added, if necessary.

In addition, by additionally adding a diluent, a dispersant, a surfactant, a binder and a lubricant, the composition of the present invention may be prepared as a formulation for injection, such as an aqueous solution, a suspension or an emulsion, pills, capsules, granules or tablets. Further, the composition of the present invention may be prepared depending on a disease or component by using a suitable method in the art or a method disclosed in Remington's Pharmaceutical Science (Mack Publishing Company, Easton Pa., 18th, 1990).

The composition of the present invention may additionally contain one or more active ingredients that exhibit the same or a similar function. The composition of the present invention includes the protein at 0.0001 to 10 wt %, and preferably 0.001 to 1 wt % with respect to the total weight of the composition.

The composition of the present invention may be administered parenterally (e.g., intravenously, subcutaneously, intraperitoneally, or locally) or orally according to a desired method, and the dosage thereof varies according to a patient's body weight, age, sex, health condition and diet, an administration time, an administration method, an excretion rate, and the severity of a disease. A daily dose of the composition of the present invention may be 0.0001 to 10 mg/ml, preferably 0.0001 to 5 mg/ml, and more preferably administered once or divided into several times a day.

The composition of the present invention may contain a vector including a polynucleotide encoding the peptide of the present invention at 0.05 to 500 mg and preferably 0.1 to 300 mg, or may contain a recombinant virus including a polynucleotide encoding the peptide of the present invention at 103 to 10¹² IU (10 to 10¹⁰ PFU) and preferably 10⁵ to 10¹⁰ IU, but the present invention is not limited thereto.

Alternatively, the composition of the present invention may contain 103 to 10¹, and preferably 10⁴ to 10⁷ cells including a polynucleotide encoding the peptide of the present invention, but the present invention is not limited thereto.

In addition, an effective dose of the composition containing a vector or cells including a polynucleotide encoding the peptide as an active ingredient may be administered at 0.05 to 12.5 mg/kg, and preferably 0.1 to 10 mg/kg, or at 103 to 10⁶ cells/kg, and preferably 10² to 10⁵ cells/kg of body weight, respectively, and may be administered two to three times a day. The present invention is not necessarily limited to the above-described composition, and may be changed according to the condition of a patient and the progression of a neurological disorder.

The composition of the present invention may further include a carrier, an excipient and a diluent, which are conventionally used in preparation of a pharmaceutical composition. The composition of the present invention may be parenterally administered, and for parenteral administration, topical application, intraperitoneal injection, intrarectal injection, subcutaneous injection, intravenous injection, intramuscular injection or intrathoracic injection is preferred, and cerebrospinal injection is the most effective. However, the present invention is not limited thereto.

The composition of the present invention may be used in the form of a preparation for external use, a suppository and a sterilized injection according to a conventional method. As the carrier, excipient and diluent that can be included in the composition, lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinylpyrrolidone, water, methyl hydroxybenzoate, propyl hydroxybenzoate, talc, magnesium stearate or mineral oil may be used. In preparation, a commonly used diluent or excipient such as a filler, a thickening agent, a binder, a wetting agent, a disintegrant or a surfactant may be used. In preparations for parenteral administration, a sterilized aqueous solution, a non-aqueous solution, a suspension, an emulsion, a lyophilizing agent, or a suppository is included. As the non-aqueous solvent or the suspension, propylene glycol, polyethylene glycol, a vegetable oil such as olive oil, or an injectable ester such as ethyl oleate may be used. As a suppository base, Witepsol, Macrogol, Tween 61, cacao butter, laurin fat or glycerogelatin may be used.

A preferable dosage of the composition of the present invention may vary according to a patient's condition and body weight, the severity of a disease, a drug type, and an administration route and duration, but may be suitably selected by a person of ordinary skill in the art. However, for a preferable effect, the composition may be daily administered at 0.0001 to 1 g/kg, and preferably 0.001 to 200 mg/kg, but the present invention is not limited thereto. The daily dose may be administered once or in divided doses. The dosage does not limit the scope of the present invention in any way.

<Experimental Methods>

1. Chemicals and Antibodies

Unless indicated otherwise, all chemicals were purchased from Sigma-Aldrich Co. LLC. The following antibodies were used for co-immunoprecipitation (Co-IP), immunoblotting (IB), or immunofluorescence assay (IFA): Aβ(6E10) monoclonal antibody (mAb, Catalog No. 803015, Biolegend Inc.), green fluorescent protein (GFP) mAb (Santa Cruz Biotechnology, Inc.), SOD1 polyclonal antibody (pAb, Catalog No. sc-11407, Santa Cruz Biotechnology, Inc.), FLAG mAb (Catalog No. F3165), SOD1 mAb (B8H10, Catalog No. MM-0070, Medimabs Inc.), Ub mAb (P4D1, Catalog No. sc-8017, Santa Cruz Biotechnology, Inc.), LC3A/B mAb (Sigma-Aldrich), γ-H2AX mAb (Catalog No. 05-636, Millipore Co.) and β-actin mAb (Catalog No. A5441, Sigma-Aldrich Co.). A Procaspase Ab sampler kit (Catalog No. 12742, Cell Signaling Technology) were used to identify a neural cell death-related protein.

2. Plasmid Construction

After RNA was extracted from a human lymphocyte and HEK293 cells using an RNeasy column (Clontech Laboratories Inc.), the RNA template was subjected to reverse transcription, thereby synthesizing primary cDNA, which was used as a template for PCR to construct a plasmid. The type of a plasmid used in the experiment is as follows.

1) As pSOD1-FLAG (WT) and pSOD1-FLAG (G93A), which are mammalian cell expression plasmids, the plasmids were designed to encode FLAG (F)-tag at the C-terminus, and SOD1-F(WT) and SOD1-F(G93A) proteins in frame.

2) As pGST-SOD1(WT) and pGST-SOD1(G93A), which are prokaryotic cell expression plasmids, the plasmids were designed to encode GST-tag at the N-terminus, and GST-SOD1(WT) and GST-SOD1(G93A) proteins in frame.

3) After the pGFP-SOD1 plasmid pSOD1-F was digested with EcoRI and ApaI, the SOD1 fragment was inserted into a pEGFP-C1 plasmid (BD Bioscience Clontech Inc.). The GFP-SOD1 protein having GFP-tag at the N-terminus was expressed from this plasmid.

4) A SOD1-expressing plasmid from which the N-terminus was excised was constructed.

5) A SOD1-expressing plasmid from which the C-terminus was excised was constructed.

6) A plasmid in which a stop codon was inserted into a specific position was constructed using QuickChange site-directed mutagenesis. In the plasmid, the 93th amino acid glycine was changed to alanine (G93A), the 4 the amino acid alanine was changed to valine (A4V), the 37^{th} amino acid glycine was changed to arginine (G37R), the 43th amino acid histidine was changed to arginine (H43R), the 85^{th} amino acid glycine was changed to arginine (G85R), and the 93^{rd} amino acid glycine was changed to cysteine (G93C).

7) A plasmid expressing isomers (aa 1-40 and 1-43) was constructed.

8) A plasmid that expresses Apple-tag at the C-terminus and Aβ-mApple in frame was constructed.

3. Cell Culture and Transfection

Human embryonic kidney cells (HEK293T), murine neuroblastoma cells (Neuro 2A, N2a) and glioma cells (neuroglioma H4) were cultured in 10% (v/v) fetal serum (FEBS)—containing Dulbecco's Modified Eagles Medium (DMEM) in a 5% CO₂ incubator at 37° C.

The DMEM is produced at “‘Life Technologies, Inc.,” and includes 50 U/mL of penicillin and streptomycin.

Approximately 40 to 50% of the H4 cells were positive for a neuron marker (neuron-specific enolase; NSE). Since the H4 cells were sensitive to an Aβ aggregate, and neural cell death resulted from the Aβ aggregate, they were used in neural cell death analysis.

The cells were seeded in a 100 mm culture dish at a density of 1.5×10⁶ cells. Fourteen hours later, a plasmid was transfected into the cells using the FuGENE® 6 transfection reagent (Promega Co.) according to the manufacturer's protocol. The cells were further cultured in a 5% CO₂ incubator for 20 to 30 hours.

4. Co-Immunoprecipitation (Co-IP)

The transfected HEK293T, H4 and N2a cells were lysed in a NETN buffer and incubated on ice for 1 hour. This solution includes 100 mM of NaCl, 1 mM of EDTA, 20 mM of Tris-HCl (pH 8.0), 0.5% (v/v) NP-40 and protease inhibitors. Among the protease inhibitors, aprotinin and leupeptin have a concentration of 2 μg/ml, and PMSF has a concentration of 0.1 mM.

1 mg of a protein extract and 1 μg of a primary antibody were mixed and cultured at 4° C. for 12 hours. The mixture was mixed with Protein G Sepharose 4 Fast Flow (GE Healthcare), and cultured at 4° C. for 1 hour, yielding a binding immune complex through centrifugation. A sample buffer was added to the precipitated Sepharose and boiled, and the buffer includes 44 mM Tris-HCl (pH 6.8), 2% SDS, 144 mM β-mercaptoethanol, 10% (v/v) glycerol, and 0.004% (v/v) bromophenol blue.

The immunoprecipitated sample was subjected to 12% SDS-polyacrylamide gel electrophoresis (SDS-PAGE) to transfer proteins to a membrane, and then immunoblotting with a FLAG antibody, a SOD1 antibody and a GFP antibody.

5. Immunoblotting (IB)

Cells were lysed in an RIPA buffer and incubated at 4° C. for 30 minutes. The RIPA buffer includes 2 μg/mL of a composition including 0.15 M NaCl, 0.5% (v/v) sodium-deoxycholate, 0.1% (v/v) SDS, 0.05 M Tris-HCl (pH 8.0), 1% (v/v) NP-40, aprotinin and leupeptin, 1 mM PMSF and a phosphatase inhibitor.

The phosphatase inhibitor included 1 mM Na₃VO₄ and 1 mM NaF. A protein concentration was measured by a Bradford assay (Bio-Rad Laboratories). The sample was separated in a 12% or 15% SDS-polyacrylamide gel.

The cells were lysed in a lysis buffer (Catalog No. 9803, Cell Signaling Technology), and the sample was separated in a 13% blue gel according to the manufacturer's protocol (MitoSciences Inc.).

Proteins were transferred to Protran® nitrocellulose membranes (Sigma-Aldrich Co. LLC), and then the membranes were incubated in a 5% non-fat dried milk/TBST solution at room temperature for 1 hour. The milk/TBST solution includes 10 mM Tris-HCl (pH 7.5), 150 mM NaCl and 0.1% Tween 20.

The membranes were incubated with a primary antibody at room temperature for 1 hour, and incubated again with a secondary antibody for 1 hour. The antigen-antibody complex was detected using an enhanced chemiluminescence kit (Santa Cruz Biotechnology, Inc.).

6. Immunofluorescence Assay (IFA)

Twenty-four hours after the transfection, N2a cells were incubated in a 4% paraformaldehyde fixation solution for 15 minutes. The cells were washed with PBS three times, and incubated in a 50 mM NH₄Cl/PBS quenching solution for 10 minutes.

Subsequently, the cells were washed with PBS three times, and incubated in a 0.1% Triton X-100/PBS solution for 5 minutes. The cells were then treated and incubated with 2% FBS/blocking buffer at room temperature for 1 hour. The cells were then treated and incubated with FLAG antibodies diluted 1/100 at room temperature for 2 hours, washed with PBS three times, and then treated and incubated with a goat anti-mouse Alexa Fluor®-red 594 dye.

The sample was washed with PBS three times, and mounted on a glass slide using a FluoroGuard Antifade reagent (Bio-Rad Laboratories, Inc.). A fluorescent image was obtained using a confocal laser scanning microscope (Zeiss LSM 510 Meta, Carl Zeiss AG), and processed using Adobe® Photoshop®, and a fluorescence signal intensity was measured using a ZEN 2009 Light Edition software program.

7. GST Pull-Down Assay

10 μl of Glutathione Sepharose 4B beads were added to an E. coli cell lysate containing a GST-SOD1 (WT or G93A) protein and incubated at 4° C. for 1 hour. After washing with PBST three times, the GST-SOD1 protein was purified.

A solution containing approximately 8 μg of the GST-SOD1 protein was mixed with guanidine-hydrochloride (Gu-HCl) to adjust a concentration to 0.6 M, and incubated at room temperature for 2 hours. Afterward, a HEK293T cell lysate (protein 2 mg) containing a GFP-SOD1 protein was added, and incubated at 4° C. for 16 hours. A GST pull-down protein complex was washed with a PBST buffer three times.

The protein complex was boiled in 20 μl of a 2X sample buffer, separated by 12% SDS-PAGE, and immune-reacted with a GFP antibody (1:5000) and a GST antibody (1:10,000), followed by analysis.

8. Quantitative Analysis of Protein Mobility Using Fluorescence Recovery after Photobleaching (FRAP)

Fluorescence recovery after photobleaching (FRAP) is a technique for detecting the mobility of a fluorescence-tagged molecule in living cells. N2a cells were cultured in a 35 mm cell culture dish at a density of 5×10⁴ cells/dish, and after 12 hours, a target plasmid was transfected into the cells.

Thirty six hours after the transfection, whether an aggregate was formed was analyzed by a laser confocal microscope. A specific area (white box part) was photobleached 15 times using 594 nm laser, and then fluorescence recovery in the photobleached part was measured at an interval of 3 seconds.

9. Formation of β-Amyloid Aggregate A monomer recombinant Aβ42 (Catalog No. 03-111, Invitrogen™ Co.) peptide was added to 1,1,1,3,3,3-hexafluoro-2-propanol (HFIP, Sigma-Aldrich Co. LLC) and stored in a −80° C. freezer, and the HFIP was evaporated and removed immediately before use. For the Aβ aggregate formation, 20 μM of Aβ peptides were dissolved in a 0.1 M HEPES/TBS buffer or 0.01 M HCl/TBS buffer and incubated at 37° C. for 14 days. The TBS buffer included 50 mM Tris-HCl and 150 mM NaCl.

10. Statistical Analysis

All data were represented as mean±SEM (standard error of the mean). When the P value was 0.05 or less, it was considered to be statistically significant. Statistical significance was evaluated through post comparison between one-way ANOVA and a Tukey's test using SPSS ver.24.0.

### EXAMPLES

### Example 1. Stability of β1-β5 Sequence of Peptide and Binding with β-Amyloid

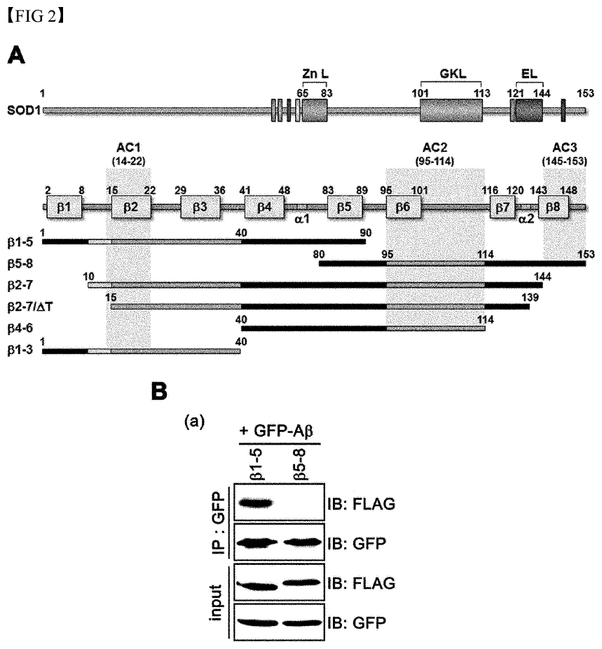

To identify a β-strand exposed at a protein surface and inhibiting Aβ protein aggregate formation through interactions with Aβ, all types of truncated fragments of SOD1 consisting of eight β-strands (β1 to β8) were systemically designed and constructed (FIG. 2). FIG. 2 schematically illustrates SOD1 and the β1-to-β8 structure of SOD1, and the sequences of Peptides 1 to 6.

To determine a determinant binding to Aβ, various peptides from which a specific β-strand was removed were constructed. Peptide 2 was prepared by removing a β-turn linked to the β2 strand in Peptide 1. The β-strand sequences of Peptides 1 to 6 are shown in Table 2 below. Peptides 1 to 6 and Aβ were overexpressed in nerve cells (N2a), and then a Co-IP assay was performed.

TABLE 2
Amino acid sequence of Peptides 1 to 6

| Peptide 1 | Peptide 2 | Peptide 3 | Peptide 4 | Peptide 5 | Peptide 6 |
|---|---|---|---|---|---|
| β 2-7 | β 2-7/T | β 4-6 | β 1-3 | β 1-5 | β 5-8 |

TABLE 3
Identification of Aβ-binding site

| | Number of | Acting with β- | Binding strength | Self- | Degree of |
|---|---|---|---|---|---|
| | amino acids | amyloid | with β-amyloid | aggregation | expression |
| Peptide 1 | 135 aa | Acting | 6 | Aggregated | 5 |
| β2-7 | | | | | |
| Peptide 2 | 130 aa | Acting | 3.5 | Aggregated | 5 |
| β 2-7/T | | | | | |
| Peptide 3 | 75 aa | Not acting | 0 | Non- | 5 |
| β 4-6 | | | | aggregated | |
| Peptide 4 | 39 aa | Acting | 1 | Non- | 1 |
| β 1-3 | | | | aggregated | |
| Peptide 5 | 90 aa | Acting | 1 | Non- | 5 |
| β 1-5 (NABi) | | | | aggregated | |
| Peptide 6 | 74 aa | Not acting | 0 | Non- | 5 |
| β 5-8 | | | | aggregated | |

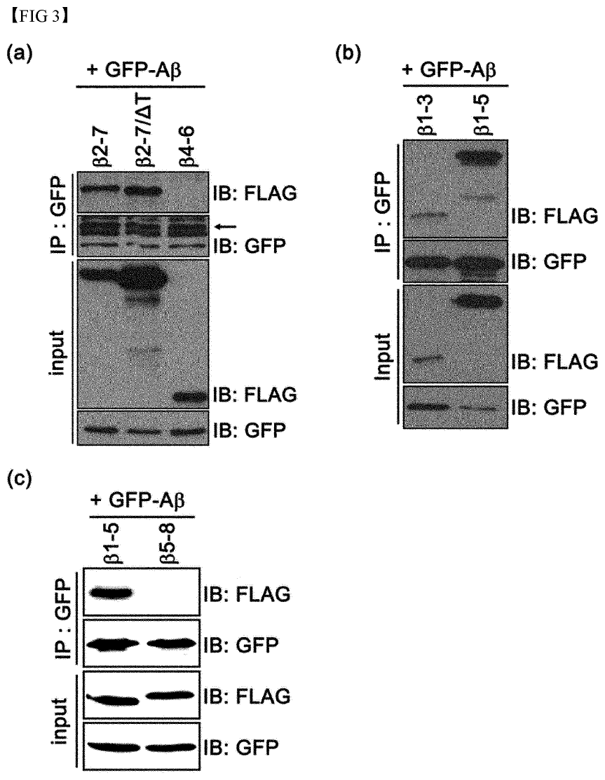

Experimental results are shown in Table 3 and FIG. 3. Referring to FIG. 3A, Peptides 1 and 2 bound to Aβ, but Peptide 3 did not bind to Aβ. Peptides 1 and 2 had a strong binding strength, but formed a self-aggregate. In Peptide 1, a self-aggregate was not formed by only removing amyloidogenic core region 2 (AC2).

Referring to FIG. 3B, Peptides 4 and 5 bound to Aβ at a similar strength. Peptide 5 expressed proteins five-fold higher than Peptide 4.

Referring to FIG. 3C, Peptide 5 bound to Aβ, but Peptide 6 did not bind to Aβ.

According to the experimental result, it can be seen that the β2-to-β3 sequence of a peptide was essential for binding to Aβ, and the β4-to-β5 sequence of a peptide was necessary for stable protein expression.

In a β-strand peptide backbone of β1 to β5, a hydrogen bond between hydrogen of an amine and oxygen of a carbonyl stabilized the peptide. The hydrogen bond played a critical role in facilitating protein folding. That is, it was shown that an energetically stable secondary structure of β2 and β3 was essential for Aβ binding.

Therefore, Peptide 5 (β1-to-β5 sequence) strongly bound to Aβ, had excellent stability, and did not form a self-aggregate, and therefore, it is suitable as an Aβ protein aggregator inhibitor, and Peptide 5 (β1-to-β5 sequence) was named NABi.

### Example 2. Stability of Peptide (NABi) of the Present Invention

To identify whether a self-aggregate of the peptide (NABi) of the present invention was formed, the peptide of the present invention was overexpressed in nerve cells (N2a), and then measured by an immunofluorescence method.

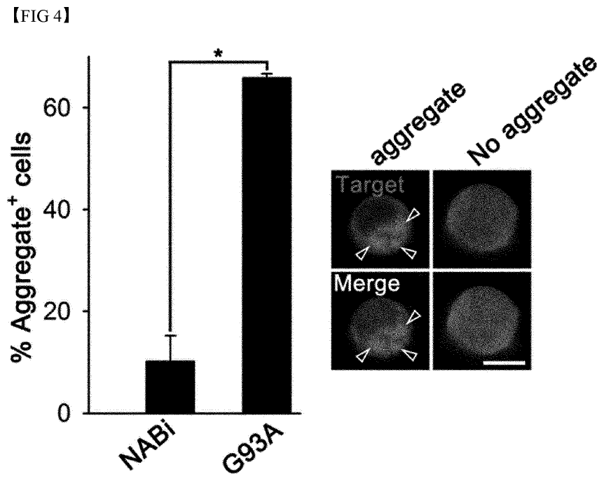

As result, protein aggregates were found in 60% or more of the G93A-expressing nerve cells. However, the peptide of the present invention had almost no protein aggregate (FIG. 4). This is because AC2 and AC3 were removed from the peptide, and therefore, a self-aggregate was not formed. Therefore, the peptide (NABi) of the present invention had an Aβ-binding determinant, and did not form a self-aggregate.

### Example 3. Verification of Cytotoxicity of Peptide of the Present Invention Through IFA

To confirm cytotoxicity of the peptide (NABi) of the present invention, the peptide of the present invention was overexpressed in nerve cells (N2a), and a nuclear morphology was observed through IFA. The nuclear morphology of a normal nerve cell (N2a), which is a control, was compared with the nuclear morphology of a nerve cell (N2a) overexpressing G93A.

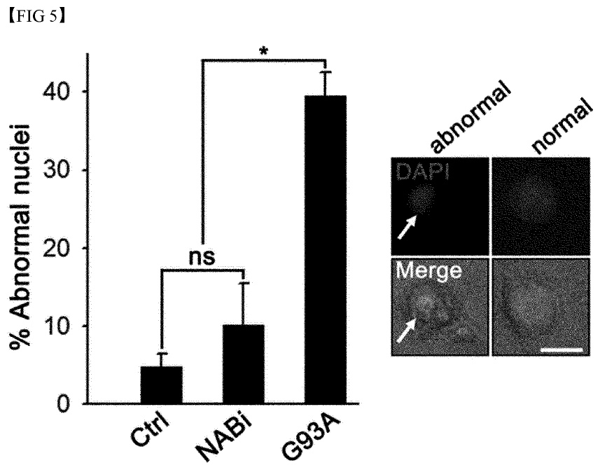

As a result, condensed and fragmented nuclei were observed in approximately 50% of the G93A-expressing cells, which means that apoptotic cell death was induced. In cells that express the peptide (NABi) of the present invention, a normal nuclear morphology, similar to that of the control, was observed (FIG. 5). This means that the peptide (NABi) of the present invention did not induce neural cell death.

### Example 4. Verification of Cytotoxicity of Peptide of the Present Invention Through IB

To confirm cytotoxicity of the peptide (NABi) of the present invention, nerve cells (N2a) were treated with staurosporine (STS) and the peptide of the present invention was expressed to evaluate the expression and activity of a neural cell death-associated protein.

For the control, normal nerve cells (N2a) were treated with STS to induce apoptotic cell death (apoptosis positive control), and then the expression and activity of a neural cell death-associated protein were evaluated.

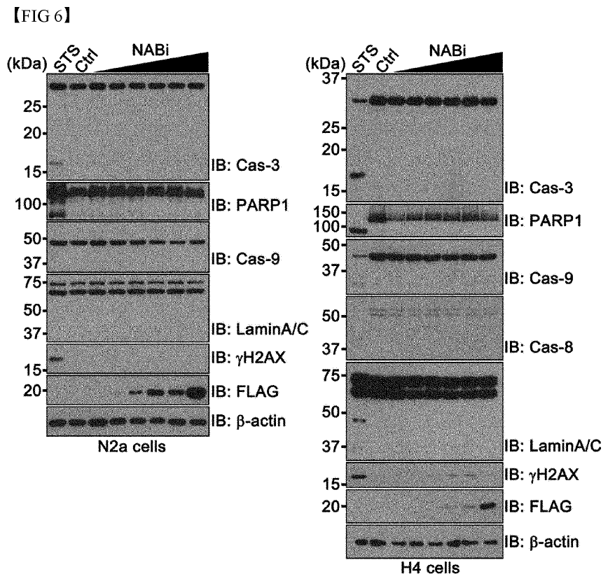

As a result, when the peptide (NABi) of the present invention was overexpressed, active caspase-3, active caspase-8, active caspase-9 and γ-H2AX were not detected. Therefore, truncated forms of a substrate of caspase-3, that is, PARP1, Lamin A/C and a substrate of caspase-6 were not detected either (FIG. 6). This means that the peptide (NABi) of the present invention did not have cytotoxicity, and apoptotic cell death was not induced.

The Lamin A/C is involved in structural stability and transcriptional regulation in the nucleus. The H2AX is used as a marker for DNA damage in a phosphorylated state.

### Example 5. Verification of β-Amyloid Aggregation Inhibition of Peptide (NABi) of the Present Invention Through IFA

To confirm the inhibition of Aβ aggregation by the peptide (NABi) of the present invention, the peptide of the present invention and Aβ were expressed in nerve cells, and then an Aβ protein aggregate was quantitatively analyzed through IFA.

For the control, Aβ was expressed in nerve cells, and then an Aβ protein aggregate was quantitatively analyzed through IFA.

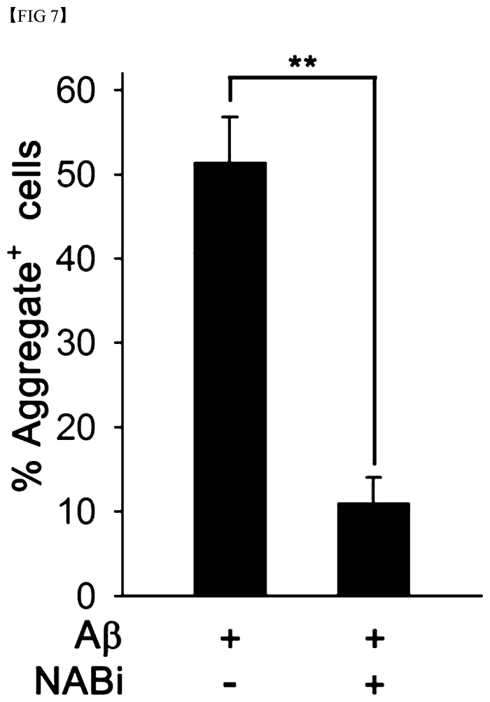

As a result, in the control, Aβ protein aggregates were observed in approximately 50% of the nerve cells. When the peptide (NABi) of the present invention was expressed, no Aβ protein aggregate was observed in 80% or more of the nerve cells (FIG. 7).

### Example 6. Verification of Inhibition of β-Amyloid Aggregation by Peptide (NABi) of the Present Invention Through IB

To confirm the inhibition of Aβ aggregation by the peptide (NABi) of the present invention, the peptide (NABi) of the present invention and Aβ were cultured under i) a 0.1 M HEPES/TBS (pH 7.4) condition or ii) a 0.01 M HCl/TBS condition, and then the degree of Aβ aggregate formation was measured through IB.

Under an Aβ aggregate formation condition, for example, i) a 0.1 M HEPES/TBS (pH 7.4) condition or ii) a 0.01 M HCl/TBS condition, the control was subjected to IB in order to measure a degree of Aβ aggregate formation.

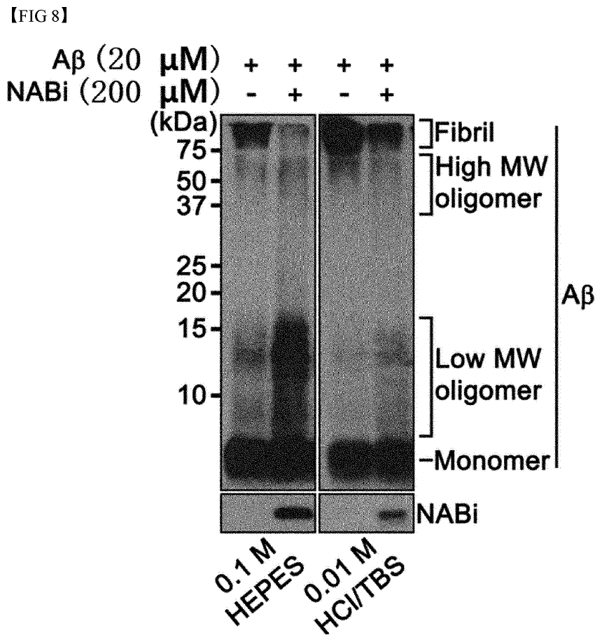

As a result, when the peptide (NABi) of the present invention was overexpressed, compared to the control, Aβ aggregates were reduced by 1/10 under the i) condition, and reduced by ⅓ under the ii) condition (FIG. 8).

This means that the peptide (NABi) of the present invention strongly binds to Aβ with high affinity, thereby effectively preventing Aβ aggregate formation.

When the peptide of the present invention binds to an Aβ monomer, the intermolecular β-sheet interactions between Aβ monomers may be prevented, and the β-sheet structure of the Aβ monomers may be destroyed or binding of the peptide (NABi) of the present invention between Aβs may be prevented due to the role of specific amino acids of the peptide (NABi) of the present invention.

### Example 7. Verification of the Inhibition of β-Amyloid Aggregation by Peptide (NABi) of the Present Invention Through FRAP Assay

To confirm the inhibition of Aβ aggregation by the peptide (NABi) of the present invention, the peptide of the present invention and Aβ were overexpressed in nerve cells (N2a), a predetermined region was repeatedly exposed to a high intensity laser beam through a FRAP assay. Afterward, an Aβ protein was removed from the laser beam-exposed region, and then fluorescence recovery according to migration of peripheral Aβ proteins was measured in real time.

For the control, after Aβ was overexpressed in nerve cells (N2a), fluorescence recovery according to migration of peripheral Aβ proteins was measured in real time through a FRAP assay.

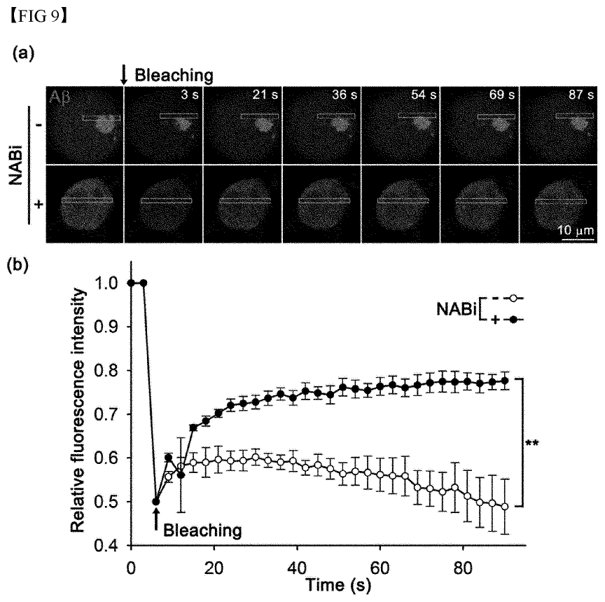

As a result, in the control, fluorescence removed by photobleaching was hardly recovered, but when the peptide (NABi) of the present invention was overexpressed, the fluorescence removed by photobleaching was recovered 50% within 20 seconds, and 75% within 90 seconds (FIG. 9). This means that Aβ aggregation was degraded and inhibited by binding of the peptide of the present invention with an Aβ aggregate.

### Example 8. Quantitative Analysis of Degree of Neural Cell Death Through PI Staining

To confirm whether the peptide (NABi) of the present invention inhibits neural cell death, the peptide of the present invention and Aβ were overexpressed in nerve cells (H4 cells), and then apoptotic cells were stained with propidium iodide (PI; dye only staining death cells).

For the control, Aβ was overexpressed in nerve cells (H4 cells).

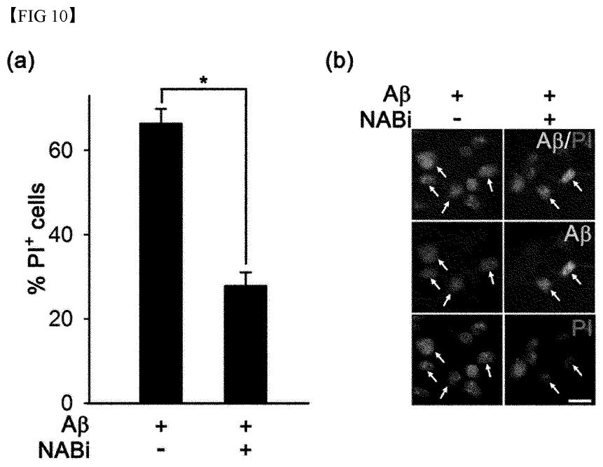

As a result, when the peptide (NABi) of the present invention was overexpressed, compared to the control, the number of the PI-stained apoptotic cells was reduced by approximately ⅓ (FIG. 10). This means that the viability of nerve cells was increased by the peptide of the present invention.

### Example 9. Quantitative Analysis of Degree of Neural Cell Death Through Nuclear Morphology

To confirm whether the peptide (NABi) of the present invention inhibited neural cell death, Aβ and the peptide (NABi) of the present invention were overexpressed, and then the number of nuclei of nerve cells (H4 cells) with a morphological change was measured.

For the control, Aβ was overexpressed in the nerve cells (H4 cells).

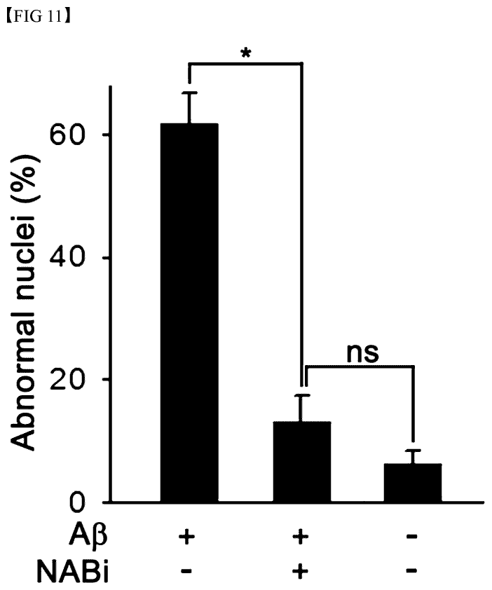

As a result, the peptide (NABi) of the present invention was overexpressed, compared to the control, there were less morphological changes in nuclei such as chromosomal condensation and fragmentation, which were at a similar level to normal cells (FIG. 11). This means that Aβ protein aggregate formation was inhibited by the peptide of the present invention, thereby reducing apoptotic cell death induced by an Aβ protein aggregate.

### Example 10. Quantitative Analysis of Neural Cell Death According to Concentration of Peptide (NABi) of the Present Invention

To confirm whether the peptide (NABi) of the present invention inhibits neural cell death, the peptide (NABi) of the present invention and Aβ were overexpressed in nerve cells (H4 cells), and then the cells were stained with AnnexinV. An expression level of the peptide of the present invention was differentially adjusted.

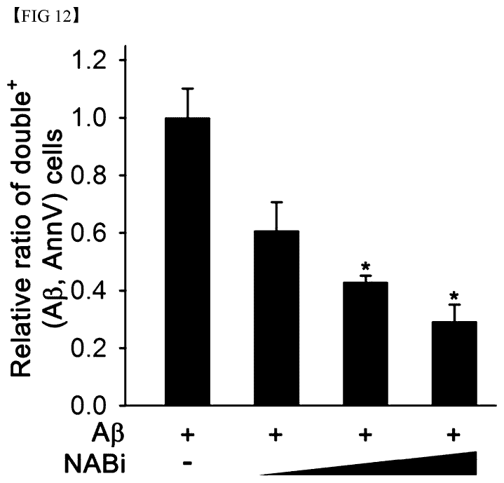

As a result, in all cases in which the peptide (NABi) of the present invention was expressed, it was seen that neural cell death was inhibited (FIG. 12). This means that the peptide of the present invention can inhibit early neural cell death (apoptotic cell death).

### Example 11. Analysis of Degree of Neural Cell Death According to Concentration of Peptide of the Present Invention at Molecular Level

To confirm whether the peptide (NABi) of the present invention inhibits neural cell death, the peptide (NABi) of the present invention and Aβ were expressed in nerve cells (H4 cells), and then the expression and activity of a neural cell death-associated protein were measured through IB. An expression level of the peptide of the present invention was differentially adjusted.

For the control, after Aβ was expressed in nerve cells (H4 cells), the expression and activity of a neural cell death-associated protein were measured through IB.

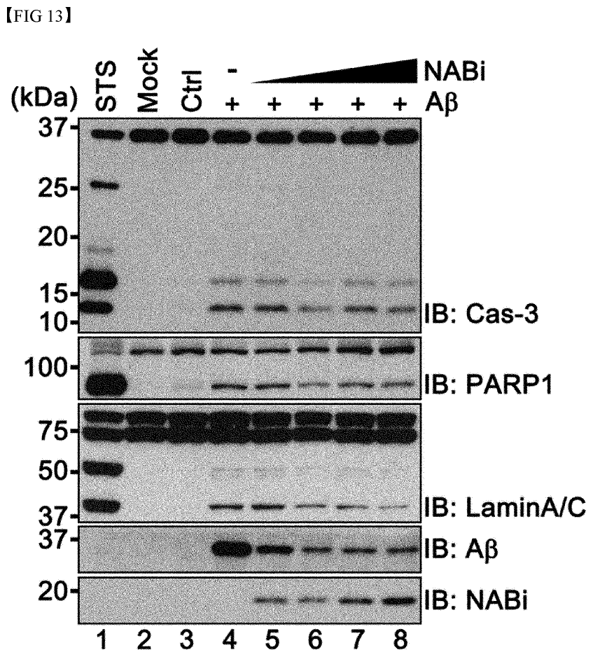

As a result, in the control, truncated caspase-3 and truncated PARP1 were detected. This means that caspase-dependent Aβ aggregate-induced neural cell death occurs (FIG. 13, lane 4). When the peptide (NABi) of the present invention was expressed, detection of truncated caspase-3, truncated PARP and truncated Lamin A/C was greatly reduced (FIG. 13, lanes 6 to 8).

The Lamin A/C is a substrate of caspase-6, and the caspase-6 plays a pivotal role in axon regeneration of nerve cells.

An ultimate purpose of the treatment of a neurodegenerative disease is for preventing neurodegeneration and neural cell death, which are induced by an Aβ protein aggregate. It was confirmed that the peptide (NABi) of the present invention inhibits neural cell death induced by Aβ. Therefore, the peptide (NABi) identified and analyzed in this study can be developed as a stable and effective therapeutic agent that can reduce neurodegeneration induced by an Aβ protein aggregate.

The forgoing detailed description illustrates the present invention. Furthermore, the above-mentioned details are for explaining the exemplary embodiments of the present invention, and the present invention may be used in various other combinations, modifications and environments. That is, alterations or modifications can be made within the scope of the inventive concept disclosed herein, the scope of equivalents to what is disclosed and/or the skill or knowledge of the art. The above-described embodiments illustrate the best mode for implementing the technical idea of the present invention, and it is also possible to make various modifications required for specific applications and uses of the present invention. Therefore, the above-detailed description of the present invention is not intended to limit the present invention. It is also to be understood that the appended claims are intended to cover such other embodiments as well.

[Sequence Listing Free Text]

```
<110> CATHOLIC UNIVERSITY INDUSTRY ACADEMIC
COOPERATION FOUNDATION
<120> Biocompatible peptideBiocompatible peptides for inhibition of
aggregation of beta-amyloid
protein
<130> PCT5036972
<150> KR 10-2017-0057419
<151> 2017-05-08
<160> 4
<170> KoPatentIn 3.0
<210> 1
<211> 30
<212> PRT
<213> Artificial Sequence
<220>
<223> peptide 1
<400> 1
Gly Asp Gly Pro Val Gln Gly Ile Ile Asn Phe Glu Gln Lys Glu Ser
1 5 10 15
Asn Gly Pro Val Lys Val Trp Gly Ser Ile Lys Gly Leu Thr
                        20 25 30
<210> 2
<211> 39
<212> PRT
<213> Artificial Sequence
<220> peptide 2
<223>
<400> 2
Ala Thr Lys Ala Val Cys Val Leu Lys Gly Asp Gly Pro Val Gln Gly
1 5 10 15
Ile Ile Asn Phe Glu Gln Lys Glu Ser Asn Gly Pro Val Lys Val Trp
                        20 25 30
Gly Ser Ile Lys Gly Leu Thr
                35
<210> 3
<211> 90
<212> PRT
<213> Artificial Sequence
<220> peptide 3
<223>
<400> 3
Ala Thr Lys Ala Val Cys Val Leu Lys Gly Asp Gly Pro Val Gln Gly
1 5 10 15
Ile Ile Asn Phe Glu Gln Lys Glu Ser Asn Gly Pro Val Lys Val Trp
                        20 25 30
Gly Ser Ile Lys Gly Leu Thr Glu Gly Leu His Gly Phe His Val His
                35 40 45
Glu Phe Gly Asp Asn Thr Ala Gly Cys Thr Ser Ala Gly Pro His Phe
        50 55 60
Asn Pro Leu Ser Arg Lys His Gly Gly Pro Lys Asp Glu Glu Arg His
65 70 75 80
Val Gly Asp Leu Gly Asn Val Thr Ala Asp
                                85 90
<210> 4
<211> 90
<212> PRT
<213> Artificial Sequence
<220>
<223> peptide 4
<400> 4
Ala Thr Lys Ala Val Cys Val Leu Lys Gly Asp Gly Pro Val Gln Gly
1 5 10 15
Ile Ile Asn Phe Glu Gln Lys Glu Ser Asn Gly Pro Val Lys Val Trp
                        20 25 30
Gly Ser Ile Lys Gly Leu Thr Glu Gly Leu His Gly Phe His Val His
                35 40 45
Glu Phe Gly Asp Asn Thr Ala Gly Cys Thr Ser Ala Gly Pro His Phe
        50 55 60
Asn Pro Leu Ser Arg Lys His Gly Gly Pro Lys Asp Glu Glu Arg His
65 70 75 80
Val Gly Asp Leu Arg Asn Val Thr Ala Asp
                                85 90
```

1. A biocompatible peptide that is derived from superoxide dismutase 1 (SOD1) and specifically binds to β-amyloid.

2. The biocompatible peptide according to claim 1, wherein the peptide is selected from the group consisting of: the β2-to-β3 amino acid sequence of SOD1; the β1-to-β3 amino acid sequence of SOD1 and the β1-to-β5 amino acid sequence of SOD1.

3. (canceled)

4. (canceled)

5. The biocompatible peptide according to claim 1, wherein the peptide consists of one or more amino acids selected from the group of amino acid sequences of SEQ ID NOs: 1 to 4.

6. The biocompatible peptide according to claim 1, wherein the peptide does not form a self-aggregate.

7. The biocompatible peptide according to claim 1, wherein the peptide does not have cytotoxicity.

8. The biocompatible peptide according to claim 1, wherein the peptide interrupts β-sheet interactions between β-amyloids.

9. The biocompatible peptide according to claim 1, wherein the peptide inhibits β-amyloid aggregation.

10. A β-amyloid aggregation inhibitor, comprising: the peptide according to claim 1.

11. A pharmaceutical composition for treating a β-amyloid aggregate-associated disease, comprising:
the peptide according to claim 1.

12. A pharmaceutical composition for treating a neurodegenerative disease, comprising:
the peptide according to claim 1.

13. The pharmaceutical composition according to claim 12, wherein the neurodegenerative disease is any one or more selected from the group consisting of amyotrophic lateral sclerosis (Lou Gehrig's disease), Parkinson's disease, Alzheimer's disease, Huntington's disease, spinocerebellar degeneration and type II diabetes.

14. An expression vector, comprising: a polynucleotide encoding the peptide according to claim 1.

15-17. (canceled)

18. A host cell that is transformed with the expression vector of claim 14.

19. The host cell according to claim 18, which is any one or more selected from the group consisting of a human embryonic kidney cell (HEK293T), a mouse neuroblastoma cell (Neuro 2A, N2a) and a glioma cell (neuroglioma H4).

20. A method for producing a peptide, comprising:
preparing an expression vector including a polynucleotide encoding a peptide specifically binding to β-amyloid;
preparing a transformant by introducing the expression vector into host cells; and
culturing the transformant to induce expression of the peptide and obtaining the peptide.

21. The method according to claim 20, wherein the peptide is derived from superoxide dismutase 1 (SOD1).

22. The method according to claim 20, wherein the peptide is selected from the group consisting of: the β2-to-β3 amino acid sequence of SOD1; the β1-to-β3 amino acid sequence of SOD1; and the β1-to-β5 amino acid sequence of SOD1.

23. (canceled)

24. (canceled)

25. The method according to claim 20, wherein the peptide consists of one or more amino acids selected from the group of amino acid sequences of SEQ ID Nos: 1 to 4.
